SEQUENCE LISTING (9 sequences)

SEQ ID NO: 1 - protein, 215 aa, synthetic construct
  REGION 1..215: note = FGF-1-linker-EGF
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSDGGGGSLENSDSECPLSHDGYCLHDGVCMYIEALDKYACNCVVGYIGERCQYRDLKWWELR

SEQ ID NO: 2 - DNA, 648 bp, synthetic construct
  misc_feature 1..648: note = FGF-1-linker-EGF
  source 1..648: mol_type = other DNA
atggctgaaggagaaatcaccaccttcacagccctgacagaaaagtttaacctgcctccagggaactacaagaagcccaaactcctctactgtagcaatggaggccacttcctgagaatccttcctgatggcactgtggatgggaccagggacaggtctgaccagcacattcagctgcagctcagtgctgaatcagtgggagaggtgtacattaagagtacagagactggccagtacttggccatggacactgatggacttctgtatggctcacagacaccaaatgaggaatgcttgttcctggaaaggctggaggagaaccattacaacacctatatctccaagaagcatgcagagaagaattggtttgttggcctcaagaagaatgggagctgcaaaagaggtcctagaactcactatggccagaaagccatcttgtttctccccctgccagtctcttctgacgggggtggaggctctctcgagaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgctag

SEQ ID NO: 3 - protein, 215 aa, synthetic construct
  REGION 1..215: note = AUT-FGF-1-linker-AUT-EGF
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAERNWFVGLKKNGSCKRGPRTHYGQKAILFLPLPVSSDGGGGSLENSDSECPLSHDGYCLHDGVCMYIEALDRYACNCVVGYIGERCQYRDLKWWELR

SEQ ID NO: 4 - DNA, 648 bp, synthetic construct
  misc_feature 1..648: note = AUT-FGF-1-linker-AUT-EGF
  source 1..648: mol_type = other DNA
atggctgaaggagaaatcaccaccttcacagccctgacagaaaagtttaacctgcctccagggaactacaagaagcccaaactcctctactgtagcaatggaggccacttcctgagaatccttcctgatggcactgtggatgggaccagggacaggtctgaccagcacattcagctgcagctcagtgctgaatcagtgggagaggtgtacattaagagtacagagactggccagtacttggccatggacactgatggacttctgtatggctcacagacaccaaatgaggaatgcttgttcctggaaaggctggaggagaaccattacaacacctatatctccaagaagcatgcagagaggaattggtttgttggcctcaagaagaatgggagctgcaaaagaggtcctagaactcactatggccagaaagccatcttgtttctccccctgccagtctcttctgacgggggtggaggctctctcgagaatagtgactctgaatgtcccctgtcccacgatgggtactgcctccatgatggtgtgtgcatgtatattgaagcattggacaggtatgcatgcaactgtgttgttggctacatcggggagcgatgtcagtaccgagacctgaagtggtgggaactgcgctag

SEQ ID NO: 5 - skipped

SEQ ID NO: 6 - DNA, 21 bp, synthetic construct
  misc_feature 1..21: note = Forward primer FGF-1 K106R
  source 1..21: mol_type = other DNA
catgcagagaggaattggttt

SEQ ID NO: 7 - DNA, 21 bp, synthetic construct
  misc_feature 1..21: note = Reverse primer FGF-1 K106R
  source 1..21: mol_type = other DNA
aaaccaattcctctctgcatg

SEQ ID NO: 8 - DNA, 27 bp, synthetic construct
  misc_feature 1..27: note = Forward primer EGF K28R
  source 1..27: mol_type = other DNA
gaagcattggacaggtatgcatgcaac

SEQ ID NO: 9 - DNA, 27 bp, synthetic construct
  misc_feature 1..27: note = Reverse primer EGF K28R
  source 1..27: mol_type = other DNA
gttgcatgcatacctgtccaatgcttc

(19) United States
(12) Patent Application Publication
(10) Pub. No.: US 2024/0156708 A1
(43) Pub. Date: May 16, 2024

(54) DUAL-FUNCTIONAL COSMETIC COMPOSITION AND PREPARING METHOD THEREOF

(71) Applicant: UBIPROTEIN CORP, Seoul (KR)

(72) Inventors: Myung-Sun KIM, Wonju-si (KR); Su Kyung OH, Yongin-si (KR); Kyunggon KIM, Seoul (KR); Kwang-Hyun BAEK, Seoul (KR); Sung-Ryul BAE, Seongnam-si (KR)

(21) Appl. No.: 18/424,838

(22) Filed: Jan. 28, 2024

Related U.S. Application Data
(63) Continuation of application No. PCT/KR2022/011256, filed on Jul. 29, 2022.

(30) Foreign Application Priority Data
Jul. 29, 2021 (KR) 10-2021-0100059

(51) Int. Cl. A61K 8/64; A61Q 19/00

(52) U.S. Cl. CPC A61K 8/64; A61Q 19/00; A61K 2800/57

(57) ABSTRACT

The present invention relates to a method for extending a half-life of a protein, comprising substituting one lysine residue in each of the amino acid sequences in two proteins, or to a protein having an extended half-life. The protein in which lysine residues have been substituted according to the present invention remains in the body for an extended period of time, and exhibits excellent treatment results. In addition, two proteins can be produced and provided in a single production process.

### CROSS-REFERENCE TO RELATED APPLICATIONS

This application is a Continuation of PCT/KR2022/011256 filed Jul. 29, 2022, which claims priority from Korean Application No. 10-2021-0100059 filed Jul. 29, 2021. The aforementioned applications are incorporated herein by reference in their entireties.

### TECHNICAL FIELD

The present invention relates to a method of preparing functional cosmetic raw materials FGF-1 and EGF into one conjugate using a biological linker. Further, the present invention relates to two functional cosmetic raw materials containing the half-life of the conjugate and a cosmetic composition containing the same.

### RELATED ART

Functional cosmetics generally refer to cosmetics with special functions other than cleaning and beauty purposes, and include products that help in whitening the skin, products that help in improving skin wrinkles, products that help in tanning the skin beautifully or protecting the skin from ultraviolet rays, and hair care products with specific functions. As raw materials used in the preparation of these functional cosmetics, an Epidermal Growth Factor (EGF) or a Fibroblast Growth Factor (FGF) has been developed and used. The EGF is known to play a role in promoting wound healing, and is widely used as a cosmetic raw material with a “skin regeneration” effect as its main function. The FGF is known as a peptide that promotes the proliferation of vascular cells or the growth of nerve cells, and is used as a cosmetic raw material with a “wrinkle improvement effect” as its main function. As such, these raw materials are used in cosmetics as anti-aging functional materials, but are expensive, have low stability, and have a short half-life to be used as concept raw materials for marketing rather than providing actual functions. Accordingly, there is a need to develop materials that have price competitiveness, high stability, and an increased half-life.

Therefore, the present inventors prepared a conjugate in which EGF and FGF-1, as representative functional cosmetic raw materials, were linked to each other with a linker, and conducted research to increase the half-life and stability of these proteins. As a result, the present inventors completed the present invention by increasing the productivity and efficiency of these functional ingredients and developing a method for producing stable dual-functional cosmetic raw materials combined with advantages of each material in a single process.

### SUMMARY

An aspect of the present invention is to provide a method of preparing functional cosmetic raw materials FGF-1 and EGF into one conjugate using a biological linkage site (linker).

Another aspect of the present invention is to provide a dual-functional cosmetic composition including the conjugate of the two proteins.

Yet another aspect of the present invention is to provide a method for extending a half-life and stability of the conjugate of the two proteins.

Yet another aspect of the present invention is to provide a cosmetic composition including the conjugate of the two proteins with extended half-life and stability.

An embodiment of the present invention provides a method for extending a half-life of a protein, a peptide, or a polypeptide, including substituting at least one lysine binding to glycine at a C-terminus of ubiquitin in the protein, the peptide or the polypeptide with arginine. In one related embodiment, the protein may be a conjugate of AUT-FGF-1 and AUT-EGF. In another related embodiment, the conjugate of AUT-FGF-1 and AUT-EGF may have an amino acid sequence of SEQ ID NO: 3 or a nucleotide sequence of SEQ ID NO: 4, in which lysine residues at position 106 from an N-terminus of FGF-1 and position 28 from an N-terminus of EGF are each substituted with arginine.

Another embodiment of the present invention provides a method for expressing and purifying FGF-1-5linkers-EGF and AUT-FGF-1-5linkers-AUT-EGF in host cells. In one related embodiment, the present invention provides an expression vector including a nucleotide sequence encoding the protein; and an optional linker, in which the promoter and the nucleotide sequence are operatively linked to each other. In yet another related embodiment, the present invention provides a host cell including the expression vector.

In the present invention, the extending of the in vivo half-life and stability of the protein is achieved by inhibiting ubiquitination of the protein. The ubiquitination is a phenomenon in which ubiquitin binds to a target protein, and has a significant impact on cellular homeostasis by acting on a protein degradation regulation mechanism. When ubiquitination of a specific protein progresses, the protein is degraded by a proteasome in the cytoplasm, which is called an ubiquitin-proteasome pathway. Most proteins that are introduced or expressed in the body are subjected to an ubiquitination process, which is known to be promoted sequentially by E1 (ubiquitin-activating), E2 (ubiquitin-conjugating), and E3 (ubiquitin-ligase) enzymes. In previous research, the present inventors developed a technology to inhibit the degradation of the protein by blocking the ubiquitin-proteasome pathway, which is referred to as an AUT™ technology. The present inventors applied the AUT™ technology to EGF (Epidermal Growth Factor) and FGF-1 (Fibroblast Growth Factor-1). Specifically, amino acid substitution was performed on the protein using the AUT™ technology to inhibit protein degradation by blocking the ubiquitination, and it was confirmed that the degradation of these proteins within the cells was reduced, and the surface charge was changed. As a result, AUT™-FGF-1 and AUT™-EGF with improved half-life and stability were obtained, and stable functional cosmetic raw materials bound with these proteins were prepared.

In the present invention, the lysine residues in the protein may be substituted with conservative amino acids. In the present invention, the “conservative amino acid substitution” means that an amino acid residue is substituted with the other amino acid residue having a side chain with a similar chemical property, for example, charge or hydrophobicity. In general, the functional properties of the protein are not substantially changed by the conservative amino acid substitution. Examples of amino acid groups having side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine and tryptophan; 5) basic side chains: lysine, arginine and histidine; 6) acidic side chains: aspartate and glutamate; and 7) sulfur-containing side chains: cysteine and methionine.

In the present invention, the lysine residue of the protein may be substituted with arginine or histidine containing a basic side chain, preferably substituted with an arginine residue.

The present invention provides a conjugate in which two proteins are linked to each other with a linker. In addition, the conjugate of the two proteins may provide extended half-life and stability by substituting one lysine residue in the amino acid sequence of these proteins with arginine. Therefore, the conjugate of the two proteins according to the present invention can be widely used as a cosmetic raw material with dual functionality.

### BRIEF DESCRIPTION OF THE DRAWINGS

FIG. 1 illustrates a schematic diagram of a conjugate of FGF-1 and EGF provided to be linked using a biological linkage site, and nucleotide sequences and amino acid sequences thereof. In FIG. 1, based on a linker sequence of GGGGS or GGG GGT GGA GGC TCT, the left side represents a FGF-1 sequence and the right side represents an EGF sequence.

FIG. 2 illustrates a schematic diagram, and nucleotide and amino acid sequences of a conjugate of AUT-FGF-1 and AUT-EGF provided to be linked using a biological linkage site. In FIG. 2, based on a linker sequence of GGGGS or GGG GGT GGA GGC TCT, the left side represents an AUT-FGF-1 sequence and the right side represents an AUT-EGF sequence.

FIG. 3 illustrates changes in half-life of each conjugate after treatment with a protein synthesis inhibitor cycloheximide (CHX).

FIG. 4 illustrates a schematic diagram, and nucleotide and amino acid sequences of a conjugate of FGF-1 and EGF expressed in E. coli.

FIG. 5 illustrates a schematic diagram, and nucleotide and amino acid sequences of a conjugate of AUT-FGF-1 and AUT-EGF expressed in E. coli.

FIG. 6 illustrates changes in expression according to culture conditions of a conjugate of AUT-FGF-1 and AUT-EGF.

FIGS. 7A and 7B illustrate states of a conjugate of AUT-FGF-1 and AUT-EGF through SDS-PAGE after purification using anion exchange chromatography and size exclusion chromatography.

FIG. 8 illustrates purification of a conjugate of AUT-FGF-1 and AUT-EGF using SDS-PAGE electrophoresis.

FIG. 9 illustrates cell proliferation efficacy of a conjugate of AUT-FGF-1 and AUT-EGF using NIH3T3 cells. For comparative treatment, conventional FGF-1 was used and a treatment concentration was 50 ng/ml.

### DETAILED DESCRIPTION

In an embodiment of the present invention, a protein is a conjugate of FGF-1 and EGF. SEQ ID NO: 1 is an amino acid sequence of the conjugate of FGF-1 and EGF provided to be linked using a biological linkage site, and SEQ ID NO: 2 is a nucleotide sequence thereof. In addition, SEQ ID NO: 3 is an amino acid sequence of a conjugate of AUT-FGF-1 and AUT-EGF provided to be linked using a biological linkage site, and SEQ ID NO: 4 is a nucleotide sequence thereof. According to Example 1, the conjugate of AUT-FGF-1 and AUT-EGF of the present invention provides an advantage of producing two proteins in one manufacturing process. In Example 2, a lysine residue at 106 position from an N-terminus of the FGF-1 amino acid sequence was substituted with an arginine residue, and a lysine residue at position 28 from an N-terminus of the EGF amino acid sequence was substituted with an arginine residue. The proteins with the substituted amino acid sequences of Example 2 above provide extended half-life and stability.

In an embodiment of the present invention, an internal restriction enzyme BamHI site was removed to prepare plasmid DNA to confirm the extended half-life. To this end, first, a nucleotide sequence encoding an arginine residue at position 39 from the N-terminus of FGF-1 was substituted from AGG to AGA, but the arginine residue was unchanged. After preparing primers using a DNA sequence to induce a specific mutation, plasmid DNA substituted with a specific nucleotide sequence was prepared by performing PCR. In another embodiment of the present invention, GGGGS (SEQ ID NO: 5) was used as a biological linkage site, a linker to link two proteins into one. The protein conjugate expressed in E. coli was repeatedly used with the five linker sequences (GGGGSGGGGSGGGGSGGGGSGGGGS). In another embodiment of the present invention, site-directed mutagenesis was used to substitute a lysine residue present in the amino acid sequence of the protein with an arginine (R) residue. In this method, after primers are prepared using a DNA sequence to induce a specific mutation, plasmid DNA substituted with a specific amino acid residue was prepared by performing PCR under specific conditions.

In the present invention, the pharmaceutical composition may be delivered to the body through various routes including oral, transcutaneous, subcutaneous, intravenous, or intramuscular administration, and may be administered as an injectable preparation. In addition, the pharmaceutical composition of the present invention may be formulated according to methods well known to those skilled in the art to achieve rapid release, delayed release, or slow release after administration according to the method. The formulation includes tablets, pills, powders, sachets, elixirs, suspensions, emulsions, solutions, syrups, aerosols, soft and hard gelatin capsules, sterile injectable solutions, and sterile packaged powders. Suitable carriers, excipients and diluents include lactose, dextrose, sucrose, mannitol, xylitol, erythritol, maltitol, starches, gum acacia, alginates, gelatin, calcium phosphate, calcium silicate, cellulose, methyl cellulose, microcrystalline cellulose, polyvinyl pyrrolidone, water, methylhydroxybenzoates, propylhydroxybenzoates, talc, magnesium stearate, and mineral oil. In addition, the formulation may further include fillers, anti-agglutinating agents, lubricating agents, wetting agents, flavoring agents, emulsifiers, preservatives, etc.

In the present invention, the singular forms include the plural forms unless clearly stated otherwise. In addition, as used herein, terms such as “configuring”, “having”, and “consisting of” are interpreted as having a similar meaning to “comprising”. In the present invention, “biologically active (poly)peptide or protein” refers to a (poly)peptide or protein that exhibits useful biological activity when administered to mammals, including humans.

Hereinafter, the present invention will be described in more detail with reference to Examples. The following Examples are just illustrative of the present invention, and the present invention is not limited to the following Examples.

### EXAMPLES

### Example 1: Confirmation of Extended Half-Life of Conjugate of Two Functional Cosmetic Raw Materials

### 1 Substitution of Lysine (K) Residue

A lysine residue was substituted with arginine (R) using site-directed mutagenesis. To this end, after primers (FGF-1 K106R FP 5′-CATGCAGAGAGGAATTGGTTT-3′ (SEQ ID NO: 6), RP 5′-AAACCAATTCCTCTCTGCATG-3′ (SEQ ID NO: 7) and EGF K28R FP 5′-GAAGCATTGGACAGGTATGCATGCAAC-3′ (SEQ ID NO: 8), RP 5′-GTTGCATGCATACC TGTCCAATGCTTC-3′ (SEQ ID NO: 9) were prepared, plasmid DNA substituted with a specific amino acid residue was prepared by performing PCR. By using pcDNA3-myc-FGF-1-linker-EGF as a template, plasmid DNA in which a lysine residue was substituted with arginine (K→R) was prepared.

### 2. Confirmation of Extended Half-Life

### (1) Cloning of Expression Vector to Confirm Extended Half-Life in Human Cells

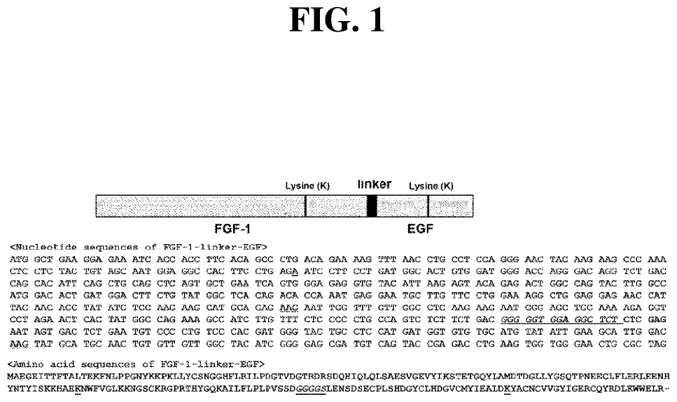

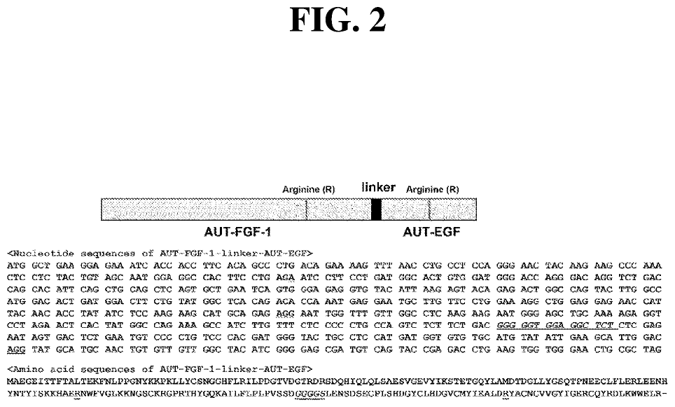

In order to develop a conjugate of two functional cosmetic raw materials, a biological linkage site (GGGGS) was used to link FGF-1 and EGF, a BamHI restriction enzyme site in FGF-1 was removed, and then pcDNA3-myc (5.6 kb) (Thermo Fisher Scientific) was fragmented with restriction enzymes BamHI and XhoI and then conjugated and cloned (FIGS. 1 and 2).

### (2) Confirmation of Extended Half-Life

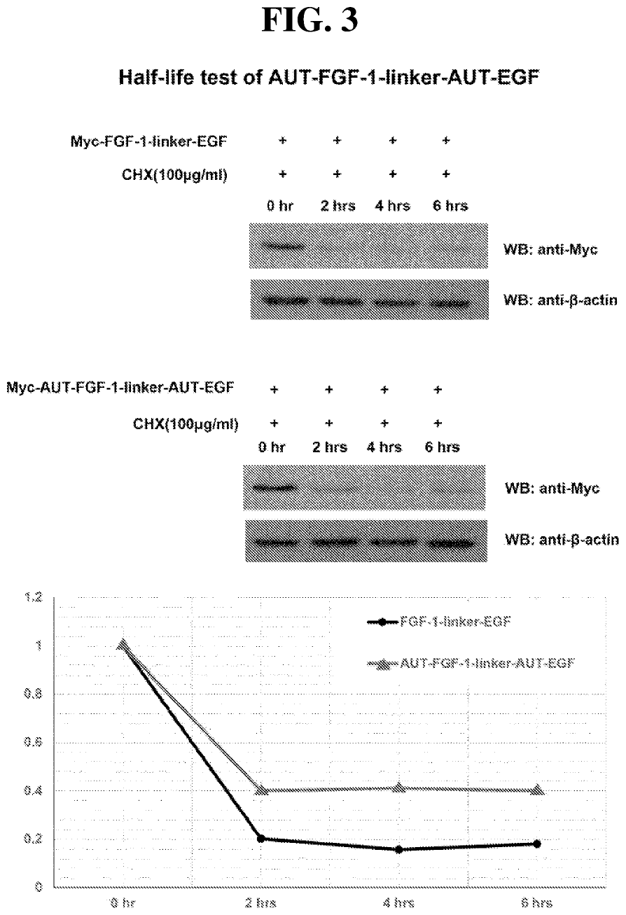

8 μg each of pcDNA3-myc-FGF-1-linker-EGF and pcDNA3-myc-AUT-FGF-1-linker-AUT-EGF were transfected into HEK293T cells (Sigma-Aldrich). 24 hours after transfection, a protein synthesis inhibitor cycloheximide (CHX) (Sigma-Aldrich) (100 μg/ml) was treated and the half-life was measured over 2 hours, 4 hours, and 6 hours. As a result, it was confirmed that the degradation of a human conjugate of AUT-FGF-1 and AUT-EGF was inhibited (FIG. 3). It was confirmed that the half-life of the human conjugate of FGF-1 and EGF was less than 1 hour, while the half-life of the human conjugate of AUT-FGF-1 and AUT-EGF was more than 2 hours, and between 2 and 6 hours, the degradation was further inhibited (FIG. 3).

### Example 2: Establishment of Expression and Purification Conditions for Conjugate of Two Functional Cosmetic Raw Materials

### 1. Expression and Purification of Conjugate

(1) Cloning of Expression Vector for Expression and Purification of Conjugate in E. coli

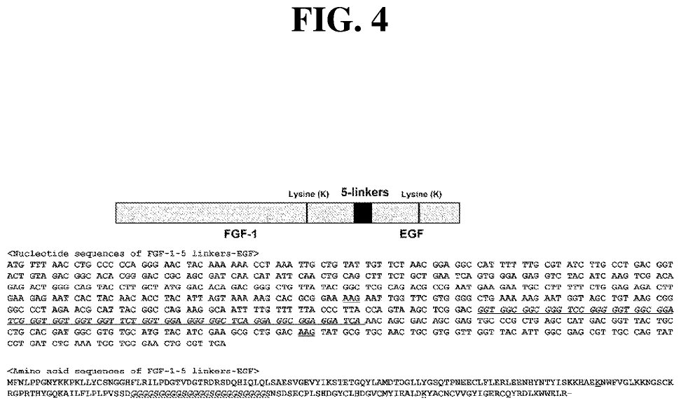

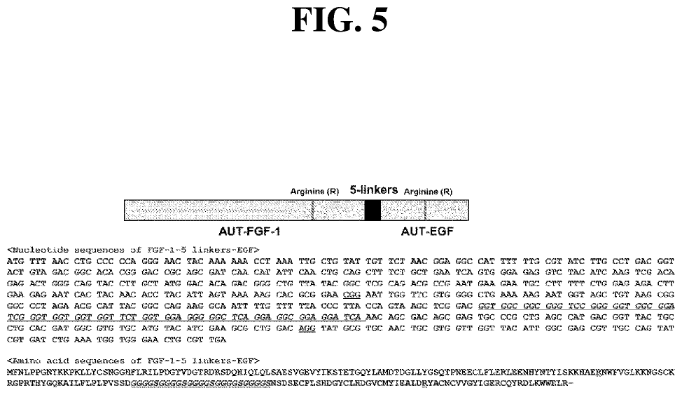

In order to link FGF-1 and EGF, a biological linkage site (GGGGS) was used repeatedly 5 times, and pET28a (5.6 kb) (Sigma-Aldrich) was fragmented with restriction enzymes NcoI and XhoI, conjugated and then cloned (FIGS. 4 and 5).

### (2) Establishment of Expression Purification Conditions

### Induction of Expression of AUT-FGF-1-5linkers-AUT-EGF

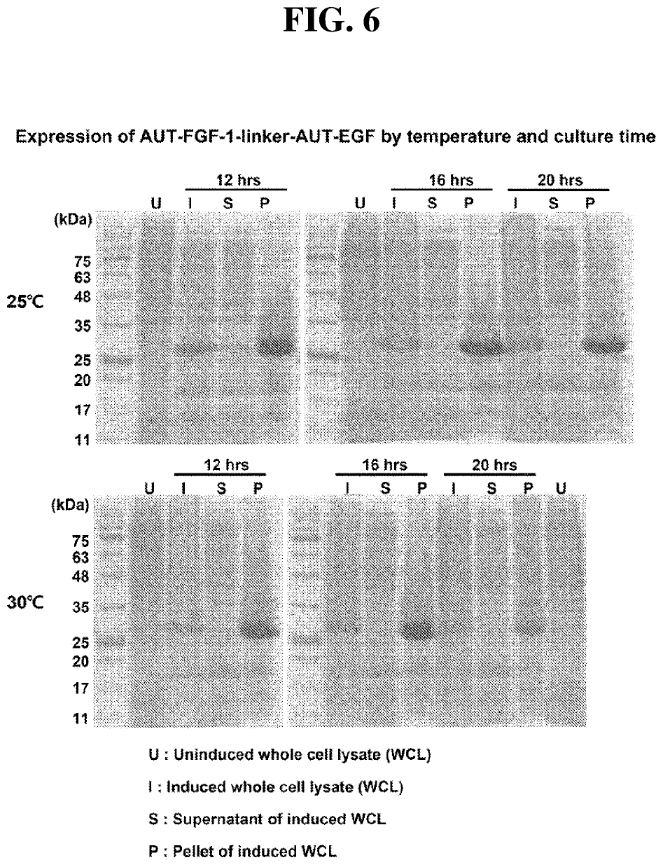

A BL21 (DE3) (Thermo Fisher Scientific) strain, transformed with a recombinant DNA encoding the amino acid sequences of FGF-1-5linkers-EGF and AUT-FGF-1-5linkers-AUT-EGF was inoculated in selective LB media containing Kanamycin and then shaking-cultured under conditions of 37° C. and 300 rpm until an OD₆₀₀ value reached 0.57. Next, 0.5 mM isopropyl-b-D-thiogalactopyranoside (IPTG) was added, and expression patterns were confirmed according to various culture conditions (FIG. 6). As the most appropriate expression conditions, shaking-culture was performed at 25° C. for 16 hours. FGF-1-5linkers-EGF was also performed in the same manner as described above.

### Cell Acquisition of AUT-FGF-1-5linkers-AUT-EGF

The culture medium was centrifuged under conditions of 3000 rpm and 4° C. for 20 minutes to obtain cell pellets. FGF-1-5linkers-EGF was also performed in the same manner as described above.

### Dissolution of AUT-FGF-1-5linkers-AUT-EGF

The cell pellets were added with 100 ml of a lysis buffer (50 mM Tris-HCl (pH 8.0), 0.5 mM EDTA, 1 mM PMSF, and 10% Glycerol) per 1 L of the culture medium and dissolved by sonication under a condition of 4° C. Insoluble pellets of the lysate were centrifuged under conditions of 13000 rpm and 4° C. for refolding, and then the obtained insoluble pellets were dissolved for 1 hour using a denaturation buffer (50 mM Tris-HCl (pH 8.0), 0.5 mM EDTA, 1 mM PMSF, 10% Glycerol, 8 M Urea), and then centrifuged under conditions of 13000 rpm and 4° C. for 15 minutes. Subsequently, the dissolved pellets were left in a refolding buffer (50 mM Tris-HC (pH 8.0), 1 mM EDTA, 1 mM PMSF, 10% Glycerol) at 4° C. for 16 hours and then centrifuged under conditions of 13000 rpm and 4° C. for 15 minutes to obtain the supernatant. FGF-1-5linkers-EGF was also performed in the same manner as described above.

### Purification of AUT-FGF-1-5linkers-AUT-EGF Using Affinity Chromatography

A Q column was mounted on AKTA and equilibrated with an equilibration buffer (50 mM Tris-HCl (pH 8.0), 0.5 mM EDTA, 1 mM PMSF, 10% Glycerol), and then applied with AUT FGF-1-5 linkers-EGF. Thereafter, the column was washed with a 6-fold amount of wash buffer (50 mM Tris-HCl (pH8.5), 0.5 mM EDTA, 1 mM PMS, 10% Glycerol, 50 mM NaCl), and then AUTFGF-1-5linkers-EGF was eluted with the same buffer as the wash buffer containing 300 mM NaCl. FGF-1-5linkers-EGF was also performed in the same manner as described above.

### Purification of AUT-FGF-1-5linkers-AUT-EGF Using Size Exclusion Chromatography

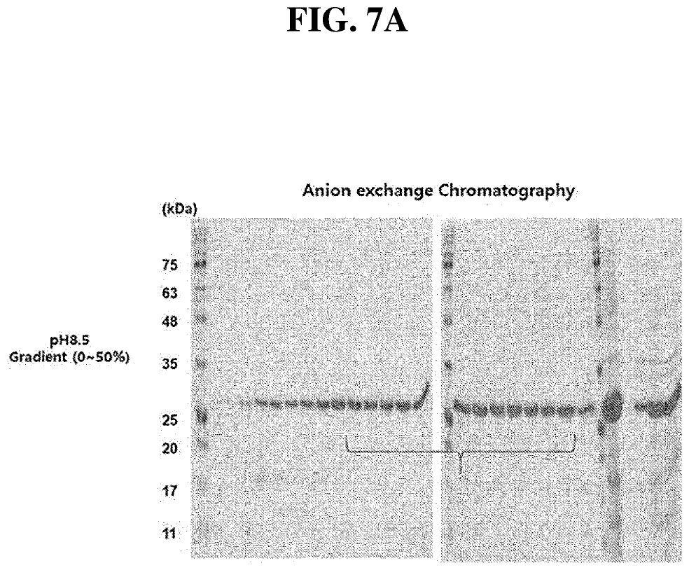

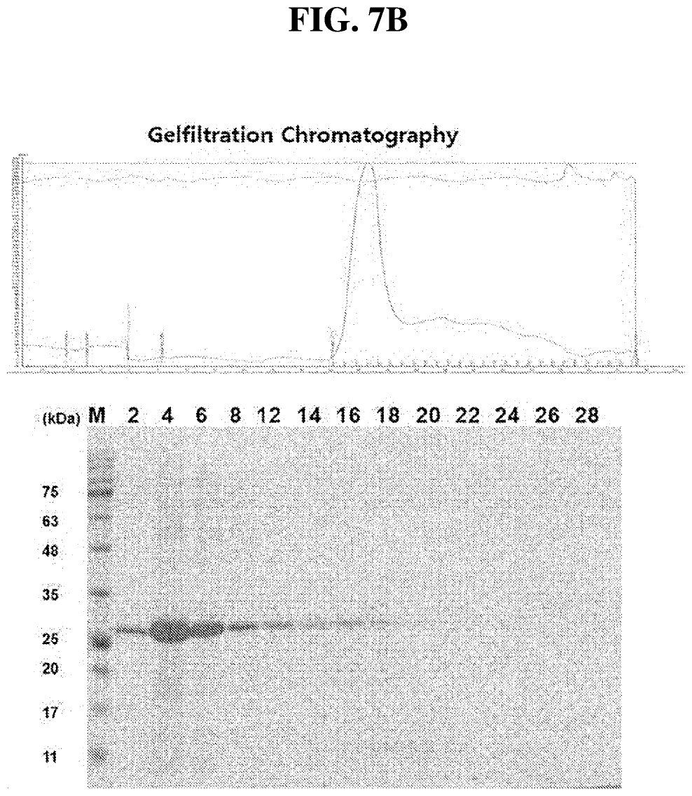

The eluent was adsorbed onto a HiPrep 16/60 Sepahacryl S-100 column equilibrated with a buffer consisting of 50 mM Tris-HCl (pH 8.0) and 400 mM NaCl for final purification. Next, the purified AUT-FGF-1-linker-AUT-EGF was dialyzed against phosphate buffered saline (pH 7.4) (FIG. 7). FGF-1-5linkers-EGF was also performed in the same manner as described above.

### Confirmation of Purity of AUT-FGF-1-5linkers-AUT-EGF

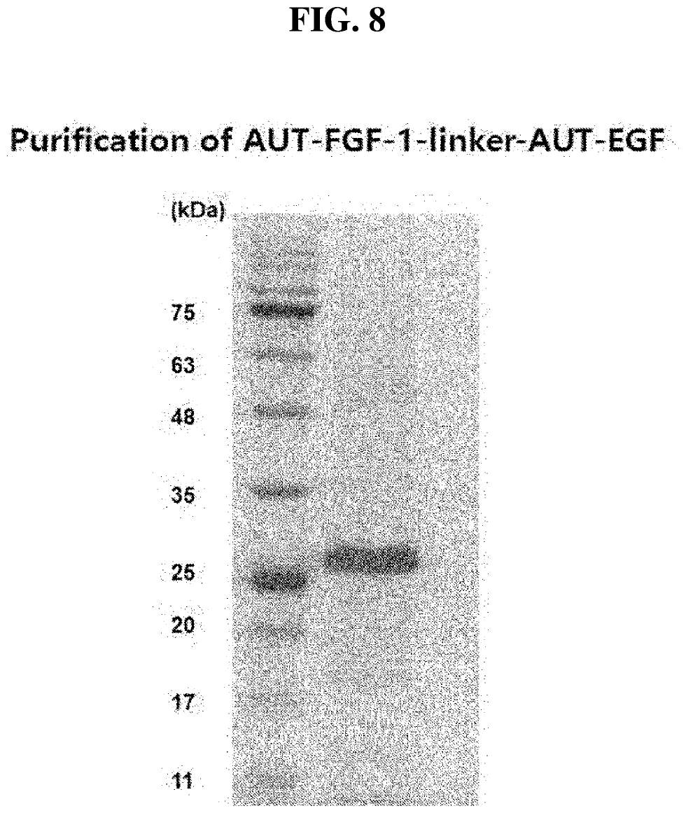

5 μg of the final purified AUT-FGF-1-linker-AUT-EGF was confirmed by SDS-PAGE (FIG. 8). FGF-1-5linkers-EGF was also performed in the same manner as described above.

### Example 3: Confirmation of Cell Proliferation Effect of Conjugate of Two Functional Cosmetic Raw Materials

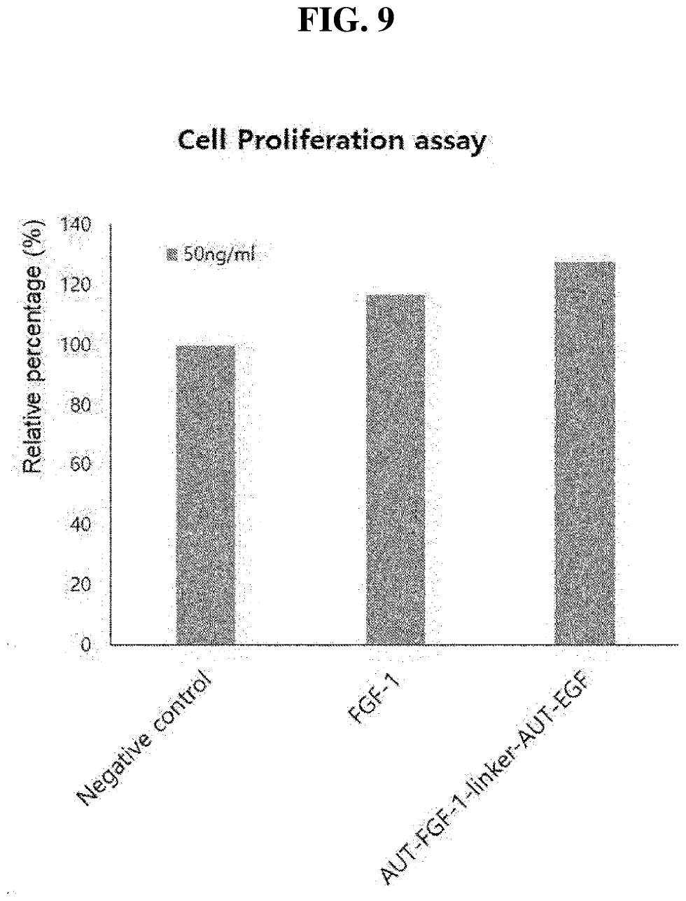

The final purified AUT-FGF-1-linker-AUT-EGF was treated with 50 ng/ml of NIH3T3 (Sigma-Aldrich) fibroblasts and the cell proliferation rate was compared and analyzed (FIG. 9). As a positive control, conventional FGF-1 was treated alone.

What is claimed is:

1. A method for extending a half-life of a protein, a peptide, or a polypeptide, comprising:
substituting at least one lysine binding to glycine at a C-terminus of ubiquitin in a protein, a peptide or a polypeptide with arginine.

2. The method for extending the half-life of the protein, the peptide, or the polypeptide of claim 1, wherein the protein is a conjugate of AUT-FGF-1 and AUT-EGF.

3. The method for extending the half-life of the protein, the peptide, or the polypeptide of claim 2, wherein the conjugate of AUT-FGF-1 and AUT-EGF has an amino acid sequence of SEQ ID NO: 1, and lysine residues at position 106 from an N-terminus of FGF-1 and position 28 from an N-terminus of EGF are each substituted with arginine.

4. A method for expressing and purifying FGF-1-5linkers-EGF and AUT-FGF-1-5linkers-AUT-EGF in host cells.

5. An expression vector comprising a promoter, a nucleotide sequence encoding AUT-FGF-1 and AUT-EGF proteins, and an optional linker, wherein the promoter and the nucleotide sequence are operatively linked to each other

6. A host cell comprising the expression vector of claim 5.

7. A cosmetic composition comprising a protein conjugate obtained according to the method of claim 4.
